(19) United States
(12) Patent Application Publication
(10) Pub. No.: US 2024/0124292 A1
(43) Pub. Date: Apr. 18, 2024

(54) AUXILIARY OPERATING DEVICE FOR DROPLET DISPENSER

(71) Applicant: Medimaging Integrated Solution, Inc., Hsinchu (TW)

(72) Inventors: SHAO HUNG HUANG, Hsinchu (TW); CHAO-TING CHEN, Hsinchu (TW); FONG HAO KUO, Hsinchu (TW); CHI-YUAN KANG, Hsinchu (TW); Chang Mu WU, Hsinchu (TW)

(21) Appl. No.: 18/486,604

(22) Filed: Oct. 13, 2023

(30) Foreign Application Priority Data
Oct. 14, 2022 (TW) 111139149

(51) Int. Cl. B67D 3/00

(52) U.S. Cl. CPC B67D 3/0077

(57) ABSTRACT

An auxiliary operation device for a droplet dispenser includes a droplet sensor, an imaging device and a processor. The droplet sensor has a detected area located between a droplet dispenser and a target area, wherein the droplet sensor detects a droplet output from the droplet dispenser, and outputs a corresponding droplet detection signal. The imaging device captures an image of the target area. The processor obtains a dripping time point at which the droplet passes through the detected area according to the droplet detection signal, and determines whether the target area is shielded within a first time range according to the image, so as to evaluate whether the droplet has successfully dropped into the target area. The above-mentioned auxiliary operating device of the droplet dispenser can objectively determine whether the droplets successfully drops into the target area, and improve the accuracy of judgment.

### BACKGROUND OF THE INVENTION

### 1. Field of the Invention

The present invention relates to an auxiliary operating device, particularly to an auxiliary operating device for a droplet dispenser.

### 2. Description of the Prior Art

The droplet dispenser is a device to drip droplets into a target area, e.g. to drip droplets of a collyrium into an eye. However, the collyrium may be hard to drip into an eye because of blinks or closures of the eye. For example, while a patient drips a collyrium by himself, the reflex-induced blinks may decrease the probability of successfully dripping the collyrium into the eye or even make the patient misjudge that the collyrium has been dripped into the eye.

Accordingly, the researchers in the field are eager to develop an auxiliary operating device that can objectively determine whether the collyrium has been successfully dripped into the eye.

### SUMMARY OF THE INVENTION

The present invention provides an auxiliary operating device for a droplet dispenser, which has an imaging device to acquire an image of a target area and determines whether the target area is shielded within an operating time range according to the image, whereby to evaluate whether the droplet has successfully dropped into the target area.

In one embodiment, the auxiliary operating device for a droplet dispenser of the present invention comprises a droplet sensor, an imaging device, and a processor. The droplet sensor has a detected area located between the droplet dispenser and a target area, wherein the droplet sensor detects a droplet output from the droplet dispenser and outputs a corresponding droplet detection signal. The imaging device captures an image of the target area. The processor is electrically connected with the droplet sensor and the imaging device. The processor obtains a dripping time point at which the droplet passes through the detected area according to the droplet detection signal and determines whether the target area is shielded within a first time range according to the image, whereby to evaluate whether the droplet has successfully dropped into the target area.

The objective, technologies, features and advantages of the present invention will become apparent from the following description in conjunction with the accompanying drawings wherein certain embodiments of the present invention are set forth by way of illustration and example.

### BRIEF DESCRIPTION OF THE DRAWINGS

The foregoing conceptions and their accompanying advantages of this invention will become more readily appreciated after being better understood by referring to the following detailed description, in conjunction with the accompanying drawings, wherein:

FIG. 1 is a diagram schematically showing an auxiliary operating device for a droplet dispenser according to one embodiment of the present invention;

FIG. 2 is a flowchart of a method of determining whether a droplet has been successfully dripped into a target area according to one embodiment of the present invention; and

FIG. 3 is a diagram schematically showing an auxiliary operating device for a droplet dispenser according to another embodiment of the present invention.

### DESCRIPTION OF THE PREFERRED EMBODIMENTS

Various embodiments of the present invention will be described in detail below and illustrated in conjunction with the accompanying drawings. In addition to these detailed descriptions, the present invention can be widely implemented in other embodiments, and apparent alternations, modifications and equivalent changes of any mentioned embodiments are all included within the scope of the present invention and based on the scope of the Claims. In the descriptions of the specification, in order to make readers have a more complete understanding about the present invention, many specific details are provided; however, the present invention may be implemented without parts of or all the specific details. In addition, the well-known steps or elements are not described in detail, in order to avoid unnecessary limitations to the present invention. Same or similar elements in Figures will be indicated by same or similar reference numbers. It is noted that the Figures are schematic and may not represent the actual size or number of the elements. For clearness of the Figures, some details may not be fully depicted.

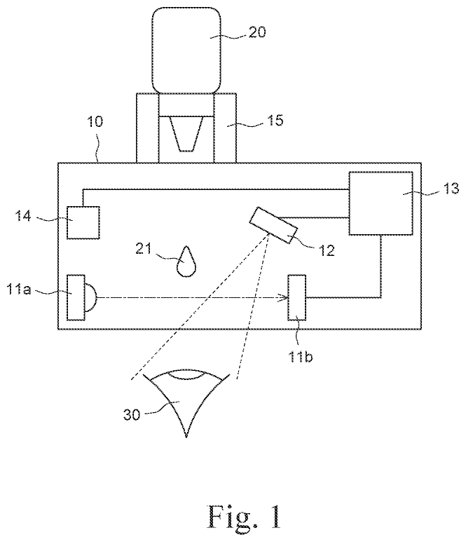

Refer to FIG. 1. In one embodiment, the auxiliary operating device 10 for a droplet dispenser of the present invention comprises a droplet sensor, an imaging device 12, and a processor 13. In one embodiment, the droplet sensor may detect whether a droplet dispenser 20 outputs a droplet 21 and output a corresponding droplet detection signal. In one embodiment, the droplet sensor includes a light source 11a and a light detector 11b. The light source 11a may provide a detecting light to illuminate a detected area. The light detector 11b detect light intensity variation generated by the process that the droplet 21 output by the droplet dispenser 20 passes through the detecting light and generates a corresponding detection signal. In one embodiment, the droplet sensor is disposed between the droplet dispenser 20 and a target area 30. In other words, the detected area of the droplet sensor at least partially overlaps the path through which the droplet 21 drips into the target area 30. In one embodiment, the target area 30 may be an eye. For example, while passing through the detected area of the droplet sensor, the droplet 21 may refract, reflect, or shield the detecting light provided by the light source 11a; thus, the intensity of the detecting light will vary; then, the light detector 11b receives the variation of the detecting light intensity and outputs a corresponding electric droplet detection signal.

The imaging device 12 may capture an image of the target area 30, such as an extraocular image of an eye. In one embodiment, the imaging device 12 at least includes an imaging lens and an image sensor. The imaging lens is disposed on a light-input side of the image sensor to form images on the image sensor. The processor 13 is electrically connected with the droplet sensor and the imaging device 12. According to the droplet detection signal output by the droplet sensor, the processor 13 may acquire a dripping time point at which the droplet 21 passes through the detected area of the droplet sensor. According to the dripping time point at which the droplet 21 passes through the detected area of the droplet sensor and the image, the processor 13 determines whether the target area 30 is shielded during a first time range. Thereby, the present invention can evaluate whether the droplet 21 successfully drops into the target area 30. The details of the evaluation method will be described below.

Refer to FIG. 1 again. In one embodiment, the auxiliary operating device 10 for a droplet dispenser of the present invention further comprises a movement detector 14, which is electrically connected with the processor 13. The movement detector 14 may detect a movement status of the auxiliary operating device 10 of the present invention and output a corresponding movement signal. For example, the movement detector 14 may detect the physical quantities of the auxiliary operating device 10 of the present invention, such as the tilt angle or vibration of the auxiliary operating device 10. According to the movement status of the auxiliary operating device 10 of the present invention, the processor 13 can evaluate whether the droplet 21 successfully drops into the target area 30. The details of the evaluation method will be described below. In one embodiment, the movement detector 14 may be an accelerometer, a gyroscope, a multiaxial sensor, a geomagnetic sensor, or a combination thereof.

Refer to FIG. 1 again. In one embodiment, the auxiliary operating device 10 for a droplet dispenser of the present invention further comprises a fixing element 15. The fixing element 15 may be connected with the droplet dispenser 20, enabling the auxiliary operating device 10 of the present invention to be fixed to the droplet dispenser 20 and securing the droplet sensor to the droplet dispenser 20 at a relative position. In a normal operation, the path, along which the droplet 21 output by the droplet dispenser 20 drips into the target area 30, will pass through the detected area of the droplet sensor. In one embodiment, the fixing element 15 may be connected with the droplet dispenser 20 in a gripping method, a locking method, a press-fitting method or a magnetic method.

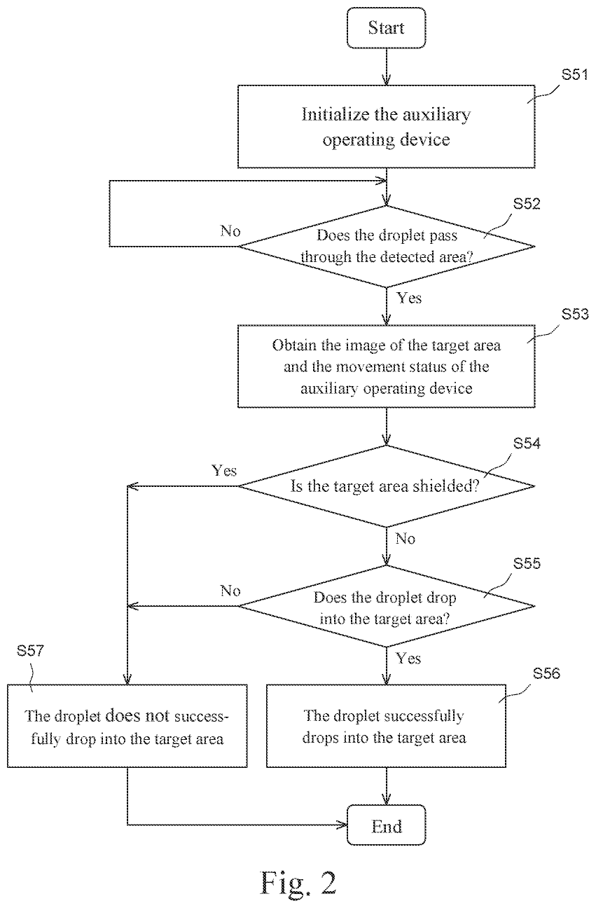

Refer to FIG. 2 for a method of determining whether the droplet 21 has successfully dropped into the target area 30. Refer to FIG. 1 also. Firstly, initialize the auxiliary operating device 10 for a droplet dispenser of the present invention (S51). At this time, the light source 11a generates the detecting light; the light detector 11b persistently receives the detecting light and converts the detecting light into an electric droplet detection signal. Besides, the imaging device 12 captures an image of the target area 30, and a movement detector 14 detects the movement status of the auxiliary operating device 10 of the present invention. Next, evaluate whether the droplet 21 passes through the detected area of the droplet sensor (S52). If the droplet 21 does not pass through the detected area of the droplet sensor, repeat Step S52 to evaluate whether the droplet 21 passes through the detected area of the droplet sensor (S52). If the droplet 21 passes through the detected area of the droplet sensor, obtain the image of the target area 30 within a specified time range, and obtain the movement status of the auxiliary operating device 10 (S53). For example, after the dripping time point at which the droplet 21 passes through the detected area of the droplet sensor, the processor 13 obtains the images of the target area 30 before and/or after the dripping time point, such as within the time interval after the droplet 21 leaves the droplet dispenser 21 and before the droplet 21 passes through the detected area, and or within the time interval after the droplet 21 passes through the detected area and before the droplet 21 reaches the target area 30; the processor 13 also simultaneously obtains the movement status of the auxiliary operating device 10 of the present invention for the judgement in succeeding steps.

Next, the processor 13 determines whether the target area 30 (such as an eye) is shielded according to the image within a first time range (S54). If the target area 30 is shielded during the dripping process (for example, the eye is closed), it is determined that the droplet 21 does not successfully drop into the target area (S57). If the target area 30 is not shielded during the dripping process (for example, the eye is opened), it is determined that the droplet 21 successfully drops into the target area 30 (S56). In one embodiment, the processor 13 determines whether the target area 30 is shielded according to whether the image presents most of the pupil or iris of the eye. It is easily understood: other methods for determining whether the eye is shielded are also applicable to the auxiliary operating device 10 of the present invention. In one embodiment, the processor 13 may use the position of the pupil or iris in the image captured by the imaging device 12 to calculate a relative position of the auxiliary operating device 10 of the present invention and the eye (the target area 30), whereby to evaluate whether the droplet successfully drops into the target area 30. For example, while the position of the pupil or iris in the image captured by the imaging device 12 is deviated from a specified area, the droplet dispenser 20 may be deviated from the target area 30, and it is thus determined that the droplet is unable to drop into the target area 30 successfully. It is easily understood: the processor 13 may only use the image of the target area 30, which is captured after the dripping time point, to perform determination, or the processor 13 may alternatively use the images of the target area 30, which are captured before and after the dripping time point, to perform determination. In one embodiment, the first time range includes the dripping time point at which the droplet 21 passes through the detected area of the droplet sensor.

Refer to FIG. 2 again. In one embodiment, while the target area 30 is not shielded during the process that the droplet 21 drips, the processor 13 further determines whether the droplet 21 drops into the target area 30 according to the movement signal output by the movement detector 14 within a second time range (S55). For example, while the tilt angle of the droplet dispenser 20 is determined to be too large or the vibration of the droplet dispenser 20 is determined to be too serious via the back calculation of the dripping time point, it is determined that the droplet 21 does not successfully drop into the target area 30 (S57); while the tilt angle or vibration of the droplet dispenser 20 is determined to be appropriate or mild, it is determined that the droplet 21 successfully drops into the target area 30 (S56). In one embodiment, the second time range is earlier than the dripping time point at which the droplet 21 passes through the detected area of the droplet sensor. However, the present invention is not limited by the abovementioned embodiment. In one embodiment, the second time range includes the dripping time point at which the droplet 21 passes through the detected area of the droplet sensor. Based on the abovementioned structure and evaluation method, the accuracy of objectively determining whether the droplet 21 successfully drops into the target area 30 is increased by about 50%, and the probability of misjudgment of users is decreased by about 25%.

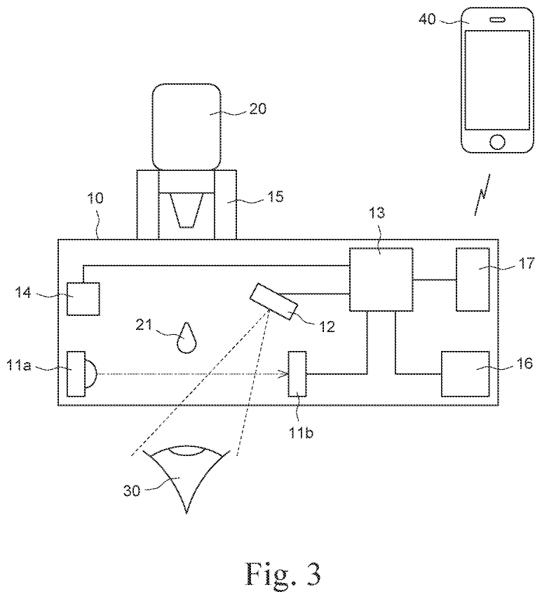

Refer to FIG. 3. In one embodiment, the auxiliary operating device 10 for a droplet dispenser of the present invention further comprises a storage element 16, in addition to the elements shown in FIG. 1. The storage element 16 is electrically connected with the processor 13, storing the dripping time points and count of the droplets 21. In one embodiment, the auxiliary operating device 10 for a droplet dispenser of the present invention further comprises a communication element 17, which is electrically connected with the processor 13. The communication element 17 enables the auxiliary operating device 10 for a droplet dispenser of the present invention to establish a communication link with an external electronic device 40, whereby the auxiliary operating device 10 can transmit the dripping time points and count of the droplets 21 to the external electronic device 40. The external electronic device 40 may be a computer, a mobile Internet-access device, a cloud server, or a dedicated medical apparatus. In one embodiment, the communication link may be a wired one or a wireless one. For example, the communication element 17 may be a universal serial bus (USB), a wired LAN (local area network) module, a Bluetooth module, a wireless LAN (WLAN) module, or a mobile communication module.

It is easily understood: the dripping time point of the droplet 21 may be regarded as the administration time; the count of droplets 21 may be regarded as the dosage. The physicians may trace the medication history of patients according to the abovementioned medication record. Therefore, the present invention can greatly favor diagnosis, therapy planning, and rehabilitation. Besides, the medication record may also assist pharmaceutical companies in accelerating the development of new medicines.

In conclusion, the present invention provides an auxiliary operating device for a droplet dispenser, which has an imaging device to acquire an image of a target area and determines whether the target area is shielded within an operating time range according to the image, whereby to evaluate whether the droplet has successfully dropped into the target area. In a preferred embodiment, the present invention further uses a movement detector to detect the movement status of the auxiliary operating device so as to assist in evaluating whether the droplet successfully drops into the target area, whereby the judgement accuracy is further increased.

While the invention is susceptible to various modifications and alternative forms, a specific example thereof has been shown in the drawings and is herein described in detail. It should be understood, however, that the invention is not to be limited to the particular form disclosed, but to the contrary, the invention is to cover all modifications, equivalents, and alternatives falling within the appended claims.

What is claimed is:

1. An auxiliary operating device for a droplet dispenser, comprising
a droplet sensor, having a detected area disposed between a droplet dispenser and a target area, wherein the droplet sensor detects a droplet output from the droplet dispenser and outputs a corresponding droplet detection signal;
an imaging device, capturing an image of the target area; and
a processor, electrically connected with the droplet sensor and the imaging device, obtaining a dripping time point at which the droplet passes through the detected area according to the droplet detection signal, and determining whether the target area is shielded within a first time range according to the image, whereby to evaluate whether the droplet has successfully dropped into the target area.

2. The auxiliary operating device for a droplet dispenser according to claim 1, further comprising
a movement detector, electrically connected with the processor, detecting a movement status of the auxiliary operating device, and outputting a corresponding movement signal, wherein the processor evaluates whether the droplet has dropped into the target area according to the movement signal within a second time range.

3. The auxiliary operating device for a droplet dispenser according to claim 2, wherein the second time range is earlier than the dripping time point.

4. The auxiliary operating device for a droplet dispenser according to claim 2, wherein the second time range includes the dripping time point.

5. The auxiliary operating device for a droplet dispenser according to claim 2, wherein the movement detector includes an accelerometer, a gyroscope, a multiaxial sensor, a geomagnetic sensor, or a combination thereof.

6. The auxiliary operating device for a droplet dispenser according to claim 1, wherein the first time range includes the dripping time point.

7. The auxiliary operating device for a droplet dispenser according to claim 1, wherein the droplet sensor includes
a light source, providing a detecting light to illuminate the detected area; and
a light detector, detecting light intensity variation, and generating the corresponding droplet detection signal.

8. The auxiliary operating device for a droplet dispenser according to claim 1, wherein the target area is an eye; the processor determines whether the eye is shielded according to a pupil or iris of the eye in the image.

9. The auxiliary operating device for a droplet dispenser according to claim 8, wherein the processor works out a relative position of the auxiliary operating device and the eye according to a position of the pupil or iris in the image to evaluate whether the droplet successfully drops into the target area.

10. The auxiliary operating device for a droplet dispenser according to claim 1, further comprising
a fixing element, connected with the droplet dispenser to enable the auxiliary operating device to be fixed to the droplet dispenser at a relative position.

11. The auxiliary operating device for a droplet dispenser according to claim 1, further comprising
a storage element, electrically connected with the processor, and used to store the dripping time points of the droplets and a count of the droplets.

12. The auxiliary operating device for a droplet dispenser according to claim 1, further comprising
a communication element, electrically connected with the processor, enabling the auxiliary operating device to establish a communication link with an external electronic device and transmit the dripping time points and a count of the droplets to the external electronic device.

13. The auxiliary operating device for a droplet dispenser according to claim 12, wherein the communication link is a wired communication link or a wireless communication link.
